(19) United States
(12) Patent Application Publication
(10) Pub. No.: US 2014/0155738 A1
(43) Pub. Date: Jun. 5, 2014

(54) APPARATUS AND METHOD FOR ULTRASOUND IMAGING

(71) Applicant: General Electric Company, Schenectady, NY (US)

(72) Inventors: Gang Cheny, Shanghai (CN); Xiaodong Han, Shanghai (CN); Jing Ye, Shanghai (CN); Michael Charles Macdonald, New Berlin, WI (US); Christopher Robert Hazard, Niskayuna, NY (US)

(73) Assignee: General Electric Company, Schenectady, NY (US)

(21) Appl. No.: 14/092,234

(22) Filed: Nov. 27, 2013

(30) Foreign Application Priority Data
Nov. 30, 2012 (CN) 201210506954.0

(51) Int. Cl. A61B 8/08

(52) U.S. Cl. CPC A61B 8/0841
USPC 600/424

(57) ABSTRACT

An apparatus comprising a convex probe, a receiver and an image generating device. The convex probe includes a number of transducer elements configured to transmit a number of sets of ultrasound signals. Each set of the ultrasound signals includes a non-steered frame and at least one steered frame angled toward the non-steered frame. The receiver is coupled to the probe and configured to receive a non-steered echo frame for the non-steered fame and at least one steered echo frame for the at least one steered frame. The image generating device is coupled to the receiver and configured to generate a non-steered image using the non-steered echo frame and a needle-enhancement image using the steered echo frame and combine the non-steered image and the needle-enhancement image into a compound image.

### BACKGROUND

Ultrasound imaging has been used for the guidance of needle insertion, such as nerve blocks, vascular access, biopsy and therapy procedures. A convex probe has been used in ultrasound imaging to transmit ultrasound signals to visualize a tissue of a patient and the needle inserted in the tissue. Each frame of the ultrasound signals includes ultrasound beams transmitted at a number of different angles into a region of tissue. Some echo beams from the needle are not in a receivable angle range of the convex probe, which causes only part of the needle to be visible. The image of the needle in the tissue is visualized as a broken line that may result in failures in localizing the needle position. The failures in localizing the needle position could result in unnecessary tissue damage and increased patient discomfort.

### BRIEF DESCRIPTION

In accordance with an embodiment of the present invention, there is provided an apparatus comprising a convex probe, a receiver, and an image generating device. The convex probe includes a number of transducer elements configured to transmit a number of sets of ultrasound signals. Each set of the ultrasound signals includes a non-steered frame and at least one steered frame angled toward the non-steered frame. The receiver is coupled to the probe and is configured to receive a non-steered echo frame for the non-steered fame and a steered echo frame for the steered frame. The image generating device is coupled to the receiver and is configured to generate a non-steered image using the non-steered echo frame and at least one needle-enhancement image using the steered echo frame. The image generating device combines the non-steered image and the needle-enhancement image into a compound image.

In accordance with an embodiment of the present invention, there is provided an apparatus. The apparatus comprises a convex probe comprising a plurality of transducer elements configured to transmit a plurality of sets of ultrasound signals, each of the plurality of sets of ultrasound signals comprising a non-steered frame and at least one steered frame angled to the non-steered frame, and a processing unit coupled to the convex probe and configured to receive ultrasound echo signals for each of the plurality of sets of ultrasound signals, generate a non-steered image and at least one needle-enhancement image using the ultrasound echo signals, and combine the non-steered image and the at least one needle-enhancement image into a compound image.

In accordance with an embodiment of the present invention, there is provided a method. The method comprises transmitting a plurality of sets of ultrasound signals from a plurality of transducer elements of a convex probe, each of the plurality of sets of ultrasound signals comprising a non-steered frame and at least one steered frame angled to the non-steered frame, receiving a non-steered echo frame for the non-steered fame and at least one steered echo frame for the at least one steered frame, generating a non-steered image using the non-steered echo frame and at least one needle-enhancement image using the at least one steered echo frame, and combining the non-steered image and the at least one needle-enhancement image into a compound image.

### DRAWINGS

These and other features and aspects of embodiments of the present disclosure will become better understood when the following detailed description is read with reference to the accompanying drawings in which like characters represent like parts throughout the drawings, wherein:

FIG. 1 is a block diagram of an ultrasound apparatus in accordance with an exemplary embodiment;

FIG. 2 is a schematic diagram of a convex probe emitting ultrasound signals in accordance with an exemplary embodiment;

FIG. 3 is a schematic diagram of the convex probe emitting the ultrasound signals in accordance with another exemplary embodiment;

FIG. 4 is a schematic diagram of the convex probe emitting the ultrasound signals in accordance with another exemplary embodiment;

FIG. 5 is a schematic diagram of the convex probe emitting the ultrasound signals in accordance with another exemplary embodiment;

FIG. 6 is a schematic diagram of the convex probe emitting the ultrasound signals in accordance with another exemplary embodiment;

FIG. 7 is a schematic diagram of the convex probe emitting the ultrasound signals in accordance with another exemplary embodiment;

FIG. 8 is a schematic diagram of the convex probe emitting the ultrasound signals in accordance with another exemplary embodiment;

FIG. 9 is a flowchart of a method of ultrasound imaging in accordance with an exemplary embodiment;

FIG. 10 is a non-steered image in accordance with an embodiment;

FIG. 11 is a needle-enhancement image in accordance with an embodiment; and

FIG. 12 is a compounding image of the non-steered image of FIG. 10 and the needle-enhancement image of FIG. 11.

### DETAILED DESCRIPTION

Unless defined otherwise, technical and scientific terms used herein have the same meaning as is commonly understood by one of ordinary skill in the art to which this disclosure belongs. The terms “first”, “second”, and the like, as used herein do not denote any order, quantity, or importance, but rather are used to distinguish one element from another. Also, the terms “a”, “an” and “one” do not denote a limitation of quantity, but rather denote the presence of at least one of the referenced items. The use of “including,” “comprising” or “having” and variations thereof herein are meant to encompass the items listed thereafter and equivalents thereof as well as additional items. The terms “connected” and “coupled” are not restricted to physical or mechanical connections or couplings, and can include electrical connections or couplings, whether direct or indirect. The term “image” broadly refers to both viewable images and data representing a viewable image.

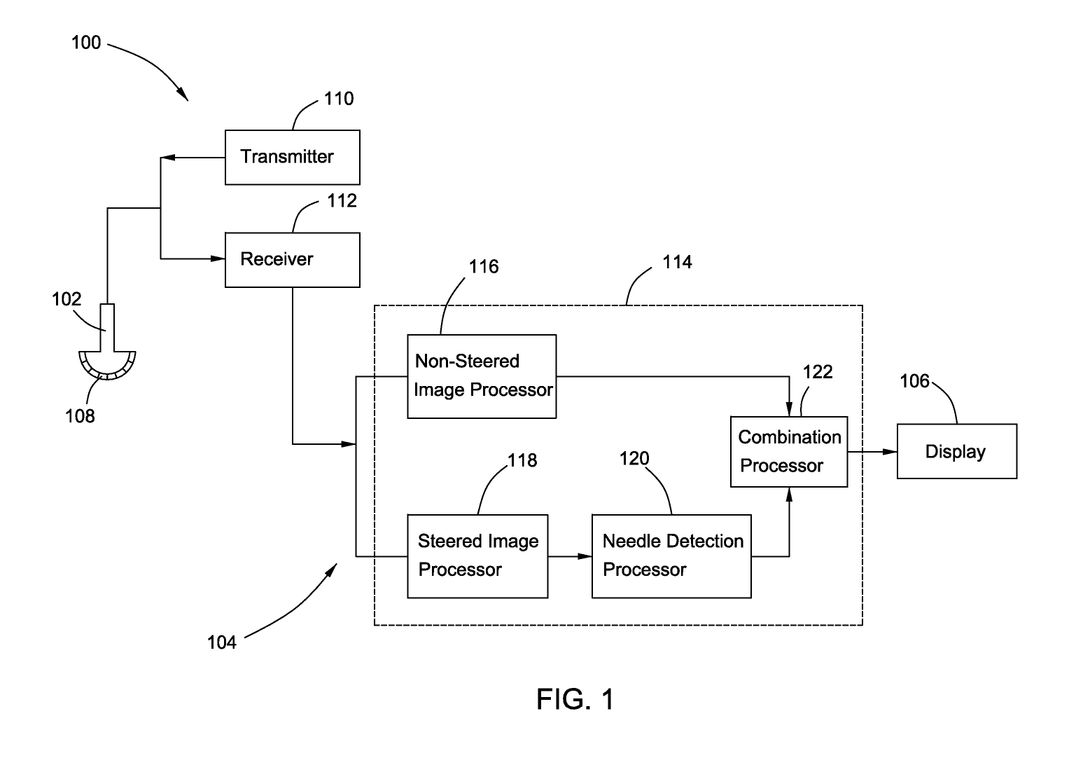

FIG. 1 illustrates a block diagram of an ultrasound apparatus 100 in accordance with an exemplary embodiment. The ultrasound apparatus 100 is configured to image a tissue of a body and guide a needle (not shown) into the body. The ultrasound apparatus 100 includes a convex probe 102, a processing unit 104 coupled to the convex probe 102 and a display device 106 coupled to the processing unit 104. The convex probe 102 is applied to a surface of the body (not shown). The convex probe 102 includes a number of transducer elements 108 configured to transmit a number of sets of ultrasound signals into the body and receive ultrasound echo signals from the body. The transducer elements 108 are arranged in a curved line along an edge of the convex probe 102. The transducer elements 108 include piezoelectric elements (not shown) that fire an ultrasound pulse. Each set of the ultrasound signals corresponds to a non-steered frame and at least one steered frame angled toward the non-steered frame. The ultrasound signals are back-scattered from structures in the body, like blood cells or muscular tissue, to produce the ultrasound echo signals.

The processing unit 104 is configured to receive the ultrasound echo signals for each set of the ultrasound signals, to generate a non-steered image and at least one needle-enhancement image using the ultrasound echo signals, and to combine the non-steered image and the needle-enhancement image into a compound image. The processing unit 104 includes a transmitter 110 coupled to the convex probe 102, a receiver 112 coupled to the convex probe 102, and an image generating device 114 coupled to the receiver 112. The transmitter 110 outputs a signal to drive the transducer elements 108 to emit the ultrasound signals. The receiver 112 receives the ultrasound echo signals from the convex probe 102 and performs amplification. The ultrasound echo signals include a non-steered echo frame for the non-steered fame and at least one steered echo frame for the at least one steered frame.

The image generating device 114 is configured to generate a non-steered image using the non-steered echo frame and at least one needle-enhancement image using the at least one steered echo frame, and to combine the non-steered image and the needle-enhancement image into a compound image. The image generating device 114 includes a non-steered image processor 116 coupled to the receiver 112, a steered image processor 118 coupled to the receiver 112, a needle detection processor 120 coupled to the steered image processor 118, and a combination processor 122 coupled to the non-steered image processor 116 and the needle detection processor 120.

The non-steered image processor 116 processes the non-steered echo frame to generate the non-steered image. The non-steered image may be a tissue image with the needle in the tissue. The steered image processor 118 generates at least one steered image using the steered echo frame. The steered image may be a tissue image with the needle in the tissue. The non-steered echo frame and the steered echo frame are individually beamformed to form a radio frequency (RF) signal. The RF signal is then demodulated to form in-phase quadrature (IQ) data pairs representative of the ultrasound echo signals. The IQ data pairs are then processed to form image frames for combining and displaying. In one embodiment, the RF signal and the IQ data pairs may be produced by the non-steered image processor 116 and/or the steered image processor 118. In another embodiment, the RF signal and the IQ data pairs may be produced by individual components separated from the non-steered processor 116 and the steered image processor 118. For example, the RF signal is produced by a beamformer (not shown) which performs beamforming and outputs the RF signal, and the IQ data pairs are produced by a RF processor (not shown) which processes the RF signal.

The needle detection processor 120 filters the steered image to remove a non-needle image from the steered image so that the needle-enhancement image remains. The non-needle image includes a tissue image, noise images and so on. The needle-enhancement image is an image with the needle only. The needle detection processor 120 may include one or more filters (not shown). The combination processor 122 combines the non-steered image and the needle-enhancement image into a compound image. The compound image is an image that has the tissue and an enhanced needle. In this embodiment, the non-steered image and the needle-enhancement image are compounded spatially. The needle image in the compound image is enhanced without degrading the quality of the compound image.

The ultrasound echo signals may be processed in real-time during a scanning session as the ultrasound echo signals are received. Additionally or alternatively, the ultrasound echo signals may be stored temporarily in a memory (not shown) during the scanning session and processed at another time or in an off-line operation. The display device 106 is coupled to the combination processor 122 and displays the compound image as a visible image. The display device 106 is configured with, for example, a graphic display or the like. The compound image displayed on the display device 106 is utilized as an image for guidance or navigation when an invasive treatment on a patient is performed.

In one embodiment, the connections among the components of the ultrasound apparatus 100 include one or more wired and/or wireless connections. For example, the display device 106 may be wirelessly connected to the processing unit 104. A wireless connection can permit the display device 106 to be remotely located from the processing unit 104. For example, the display device 106 may be located in an emergency room or surgery suite while one or more remaining components of the ultrasound apparatus 100 are located in another room, suite or building.

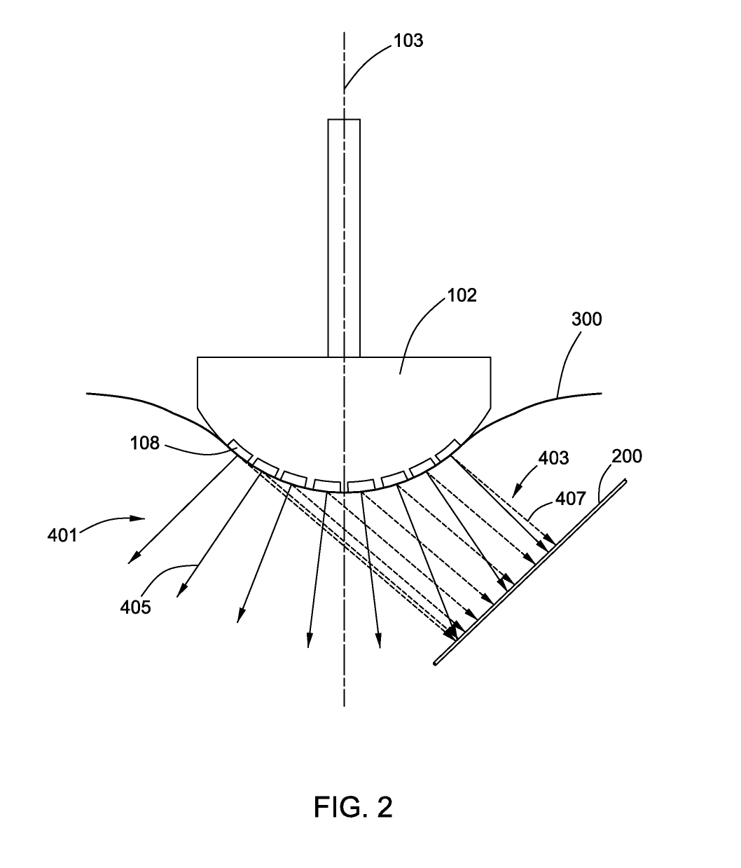

FIG. 2 illustrates a schematic diagram of the convex probe 102 emitting the ultrasound signals into the body in accordance with an exemplary embodiment. The convex probe 102 is in touch with the surface 300 of the body and a needle 200 is inserted into the body. The convex probe 102 is driven by the transmitter 110 shown in FIG. 1 to emit the ultrasound signals. Each set of the ultrasound signals shown in FIG. 2 includes a non-steered frame 401 and a steered frame 403. The non-steered frame 401 includes non-steered beams 405 each emitted by the transducer elements 108 of the convex probe 102. The non-steered beams 405 are transmitted at different angles with respect to an axis 103 of the convex probe 102. The non-steered frame 401 scans a partial fan-shaped two-dimensional (2D) region. The non-steered frame 401 may be a B-mode frame.

The steered frame 403 includes steered beams 407 steered in parallel. In this embodiment, the needle 200 is positioned near a left side of the convex probe 102 viewed from the convex probe 102. Hereinafter, left and right are directions viewed from the convex probe 102. The steered beams 407 are steered leftward with respect to the axis 103 of the convex probe 102. In another embodiment, the needle 200 is positioned near a right side of the convex probe 102, and the steered beams 407 are steered rightward with respect to the axis 103 of the convex probe 102. The needle 200 reflects the steered beams 407 which impinge upon the needle 200.

In accordance with an embodiment, the steered beams 407 impinge upon the needle 200 at a 90 degree angle, so that steered echo beams from the needle 200 are reflected along directions of the steered beams 407, thus the steered echo beams from the needle 200 may be received by the transducer elements 108. Where 90 degree angles are not achievable, this angle is as close as practicable to a 90 degree angle. The convex probe 102 emits the non-steered frame 401 and the steered frame 403 alternately.

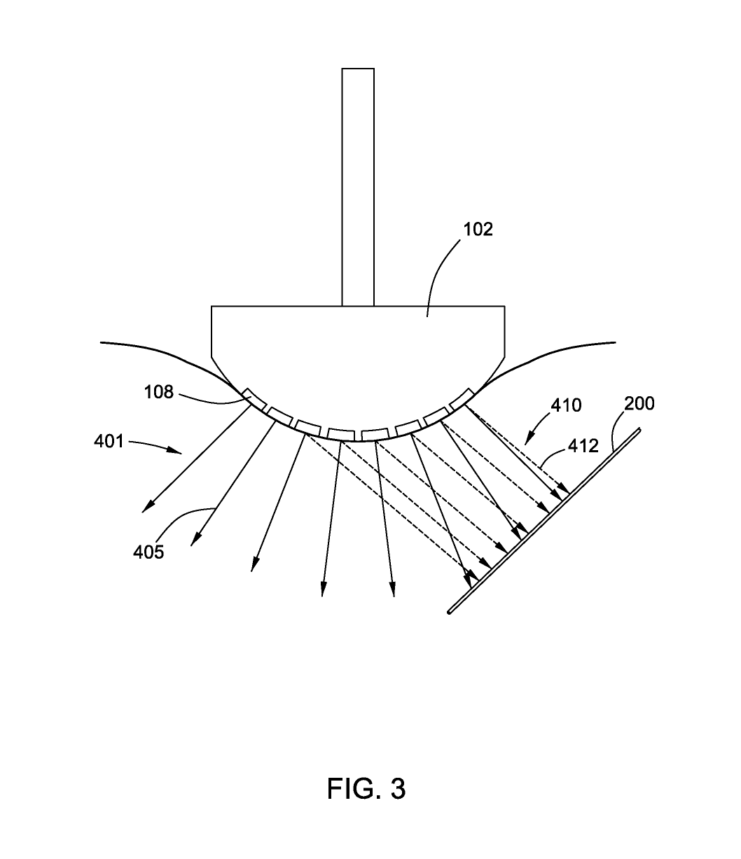

FIG. 3 illustrates a schematic diagram of the convex probe 102 emitting ultrasound signals into the body in accordance with another exemplary embodiment. In this embodiment, each set of the ultrasound signals includes the non-steered frame 401 and a steered frame 410. The ultrasound signals in this embodiment are similar to the ultrasound signals of FIG. 2. One of the differences of the ultrasound signals in FIG. 3 from the ultrasound signals in FIG. 2 is that the number of steered beams 412 of the steered frame 410 is less than that of the non-steered beams 405 of the non-steered frame 401. The number of the steered beams 412 may be reduced without substantial reduction in the needle-enhancement image quality.

In this embodiment, the steered beams 412 are steered toward the needle 200, and one or more of the transducer elements 108 which are far from the needle 200 do not emit the steered beams 412. The farther the transducer element 108 is from the needle 200, the larger a steered angle between the steered beam 412 and the non-steered beam 405 is. The larger the steered angle is, the weaker the ultrasound pulses of the steered beam 412. The transducer elements 108 near the needle 200 are used to emit strong steered beams 412 to produce strong steered echo beams, so as to speed up the image acquisition without degrading the quality of the needle-enhancement image.

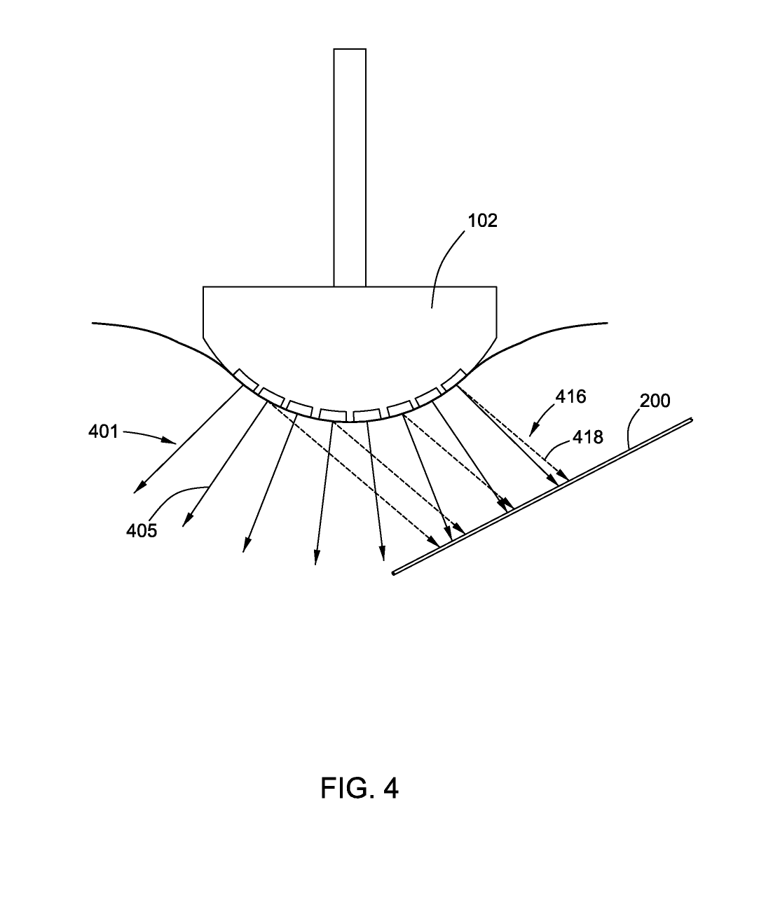

FIG. 4 illustrates a schematic diagram of the convex probe 102 emitting ultrasound signals into the body in accordance with another exemplary embodiment. Each set of the ultrasound signals includes the non-steered frame 401 and a steered frame 416. The ultrasound signals in this embodiment are similar to the ultrasound signals of FIG. 2. One of the differences of the ultrasound signals in FIG. 4 from the ultrasound signals in FIG. 2 is that the number of steered beams 418 of the steered frame 416 is less than that of the non-steered beams 405 of the non-steered frame 401. In this embodiment, the steered beams 418 of the steered frame 416 have low beam density to speed up the image acquisition. The beam density of the steered beams 418 may be half or less than half of that of the non-steered beams 405.

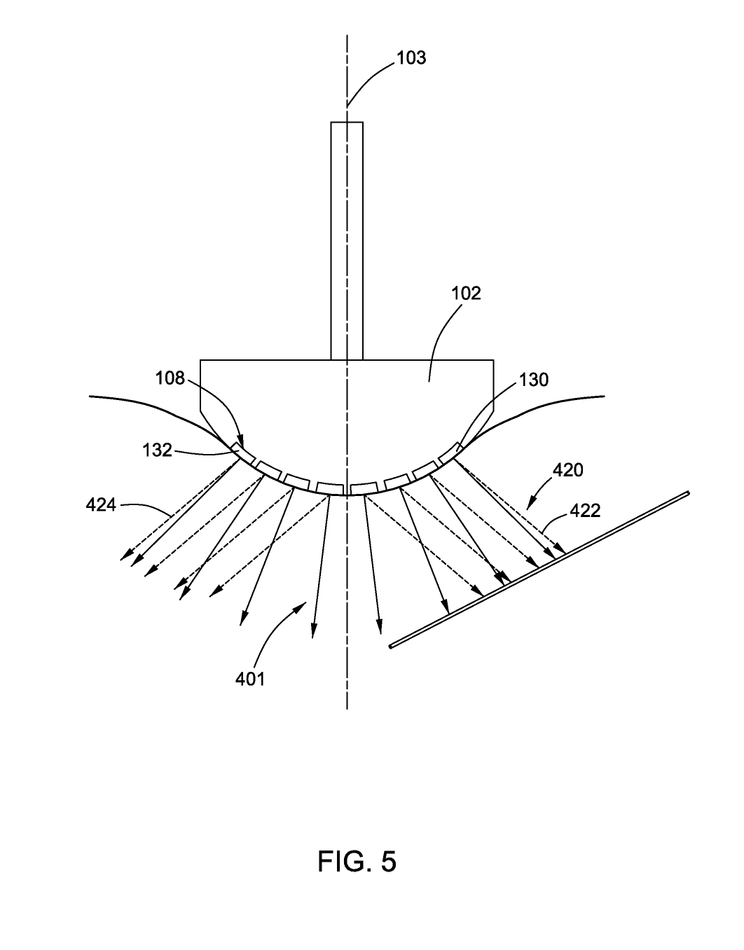

FIG. 5 illustrates a schematic diagram of the convex probe 102 emitting ultrasound signals into the body in accordance with another exemplary embodiment. Each set of the ultrasound signals includes the non-steered frame 401 and a steered frame 420. The steered frame 420 includes left steered beams 422 steered leftward with respect to the axis 103 of the convex probe 102 and right steered beams 424 steered rightward with respect to the axis 103 of the convex probe 102. The left steered beams 422 are steered in parallel and the right steered beams 424 are steered in parallel. The transducer elements 108 are arranged symmetrically according to the axis 103 of the convex probe 102. The transducer elements 108 include left transducer elements 130 arranged at the left side of the axis 103 and the right transducer elements 132 arranged at the right side of the axis 103. In this embodiment, the left transducer elements 130 emit the left steered beams 422 and the right transducer elements 132 emit the right steered beams 424, so that the left steered beams 422 and the right steered beams 424 are strong. The steered frame 420, in this embodiment, facilitates detecting the needle 200 that may be inserted from the left side of the convex probe 102 or the right side of the convex probe 102.

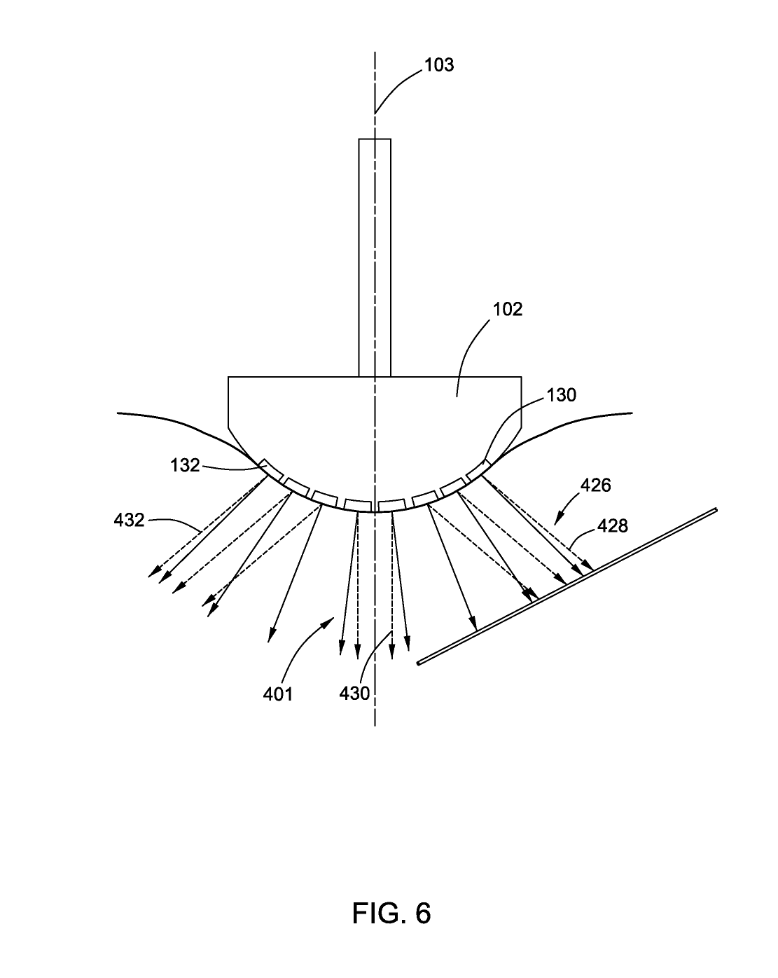

FIG. 6 illustrates a schematic diagram of the convex probe 102 emitting ultrasound signals into the body in accordance with another exemplary embodiment. Each set of the ultrasound signals includes the non-steered frame 401 and a steered frame 426. The steered frame 426 includes left steered beams 428 steered leftward with respect to the axis 103 of the convex probe 102, vertical steered beams 430 parallel with the axis 103 of the convex probe 102, and right steered beams 432 steered rightward with respect to the axis 103 of the convex probe 102. The left steered beams 428 are steered in parallel, the vertical steered beams 430 are steered in parallel and the right steered beams 432 are steered in parallel. The left steered beams 428 are emitted by some of the left transducer elements 130. The right steered beams 432 are emitted by some of the right transducer elements 132. The vertical steered beams 430 are transmitted between the left steered beams 428 and the right steered beams 432. The steered frame 426, in this embodiment, may scan a broad region to facilitate detecting the needle 200 that is inserted deeply into the body.

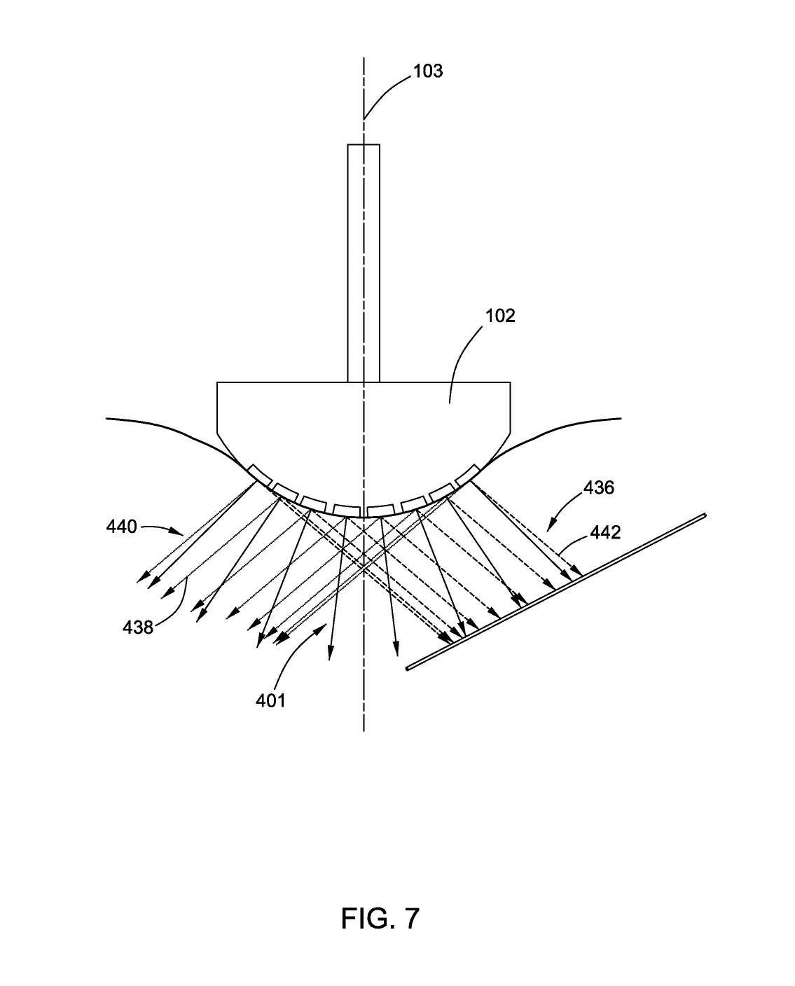

FIG. 7 illustrates a schematic diagram of the convex probe 102 emitting ultrasound signals into the body in accordance with another exemplary embodiment. Each set of the ultrasound signals includes the non-steered frame 401, a left steered frame 436 and a right steered frame 440. The left steered frame 436 is steered leftward with respect to the axis 103 of the convex probe 102. The right steered frame 440 is steered rightward with respect to the axis 103 of the convex probe 102. In this embodiment, left steered beams 442 of the left steered frame 436 are steered in parallel and right steered beams 438 of the right steered frame 440 are steered in parallel. In some embodiments, the left steered frame 436 may be the steered frame 403 of FIG. 2, the steered frame 410 of FIG. 3 or the steered frame 416 of FIG. 4, and the right steered frame 440 may have similar features with the left steered frame 436. In some embodiment, the ultrasound signals may further include a vertical steered frame (not shown) steered parallel with the axis 103 of the convex probe 102.

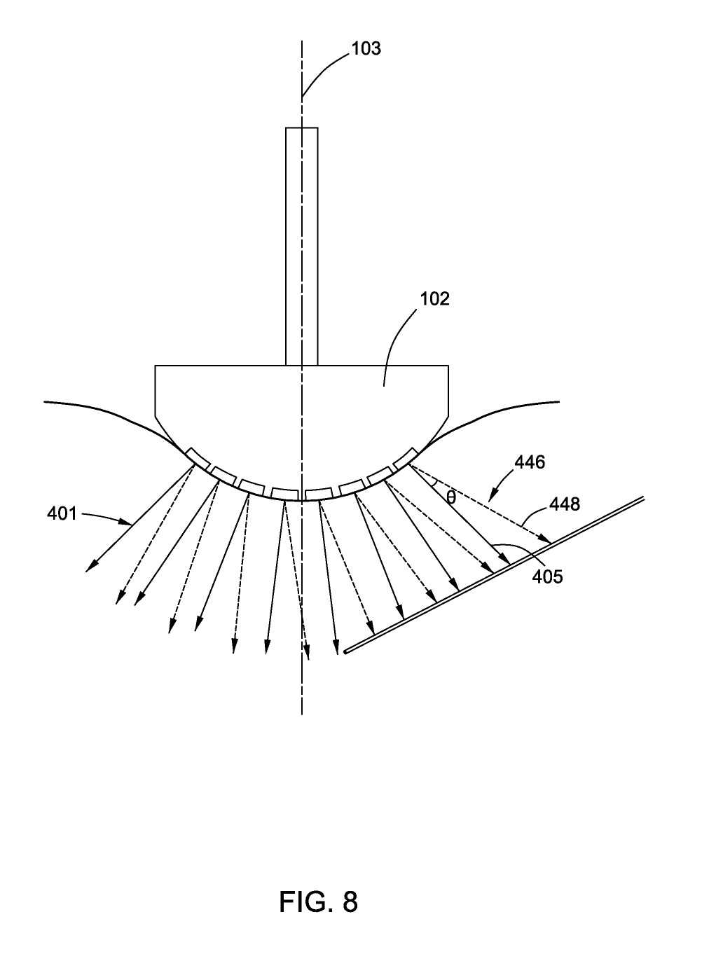

FIG. 8 illustrates a schematic diagram of the convex probe 102 emitting ultrasound signals into the body in accordance with another exemplary embodiment. Each set of the ultrasound signals includes the non-steered frame 401 and a steered frame 446. The steered frame 446 includes steered beams 448 steered at different angles with respect to the axis 103 of the convex probe 102. In this embodiment, the steered beams 448 are steered at the same angle θ with respect to corresponding non-steered beams 405. The angle θ may be 15 degrees, 30 degrees, 45 degrees or any of other degrees according the particular application. The steered beams 448 may be steered leftward or rightward with respect to the non-steered beams 405. The steered frame 446, in this embodiment, may scan a partial fan-shaped two-dimensional (2D) region.

The number of steered frames is not fixed. In certain embodiments, the ultrasound signals may include one or more of the steered frames 403, 410, 416, 420, 426, 436, 440 and 446 in FIGS. 2 to 8, and/or one or more of other forms of steered frames. Each of the steered echo frames for the steered frames may be processed to produce one needle-enhancement image. A transmit frequency for the steered frame 403, 410, 416, 420, 426, 436, 440, 446 in FIGS. 2 to 8 may be set lower than that of the non-steered frame 401 to produce strong steered echo beams having less attenuation.

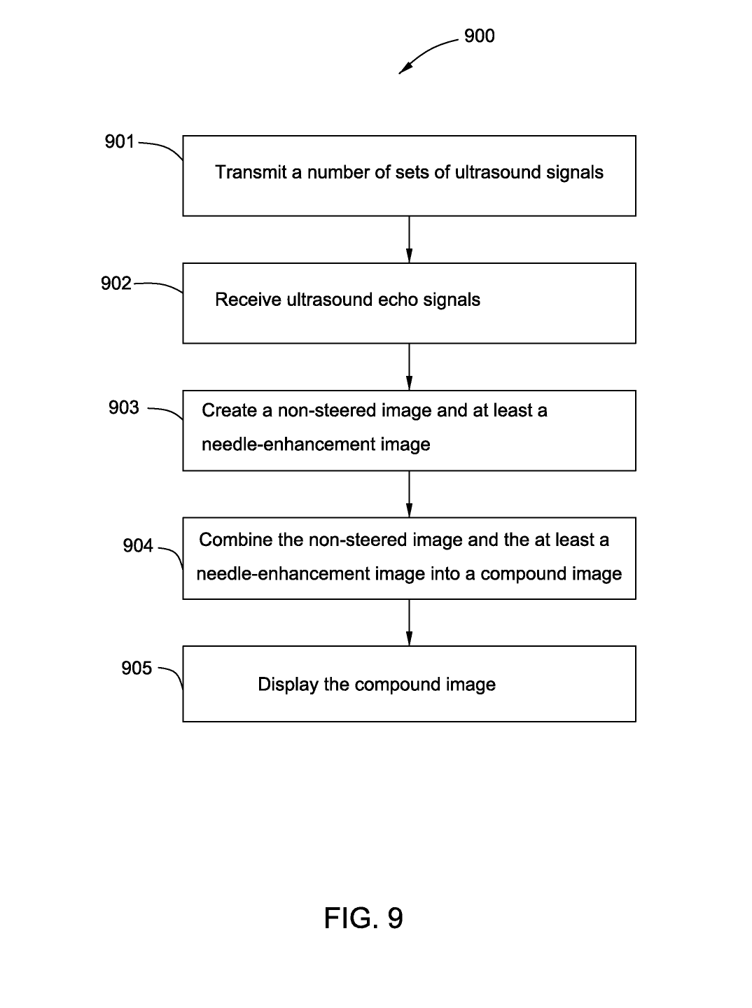

FIG. 9 illustrates a flowchart of a method 900 of ultrasound imaging in accordance with an exemplary embodiment. While the actions of the method 900 are illustrated as functional blocks, the order of the blocks and the separation of the actions among the various blocks shown in FIG. 9 are not intended to be limiting. For example, the blocks may be performed in a different order and an action associated with one block may be combined with one or more other blocks or may be sub-divided into a number of blocks.

The method 900 includes steps 901-905. At step 901, a number of sets of ultrasound signals are transmitted from transducer elements of a convex probe. Each set of the ultrasound signals includes a non-steered frame and at least one steered frame angled toward the non-steered frame. For example, in FIG. 1, the transducer elements 108 of the convex probe 102 are driven by the transmitter 110 to emit the ultrasound signals. Each set of the ultrasound signals may include the non-steered frame 401 and one or more of the steered frames 403, 410, 416, 420, 426, 436, 440, 446 in FIGS. 2 to 8.

At step 902, ultrasound echo signals for the ultrasound signals are received. Each set of the ultrasound echo signals includes a non-steered echo frame for the non-steered fame and at least one steered echo frame for the steered frame. For example, the non-steered frame 401 and the steered frames 403, 410, 416, 420, 426, 436, 440, 446 in FIGS. 2 to 8 are back-scattered from the structures in the body and/or the needle inserted in the body to produce the ultrasound echo signals. The receiver 112 shown in FIG. 1 receives the ultrasound echo signals.

At step 903, a non-steered image is generated using the non-steered echo frame and at least one needle-enhancement image is created using the steered echo frame. For example, in FIG. 1, the non-steered image processor 116 processes the non-steered echo frame to generate the non-steered image, and the steered image processor 118 and the needle detection processor 120 process the steered echo frame to generate the needle-enhancement image. One steered echo frame is processed to produce one needle-enhancement image. The step 903, at which at least one needle-enhancement image is generated, includes generating at least one steered image using the steered echo frame and selecting the needle-enhancement image from the steered image. For example, in FIG. 1, the steered image processor 118 generates the steered image using the steered echo frame, and the needle detection processor 120 filters the steered image to remove a non-needle image from the steered image so as to make sure only the needle is enhanced.

At step 904, the non-steered image and the needle-enhancement image are combined into a compound image. For example, in FIG. 1, the combination processor 122 combines the non-steered image and the needle-enhancement into the compound image, so that the needle is enhanced while the quality of the tissue image is not degraded. At step 905, the compound image is displayed. For example, the display device 106 shown in FIG. 1 displays the compound image as a visible image for the guidance of needle insertion.

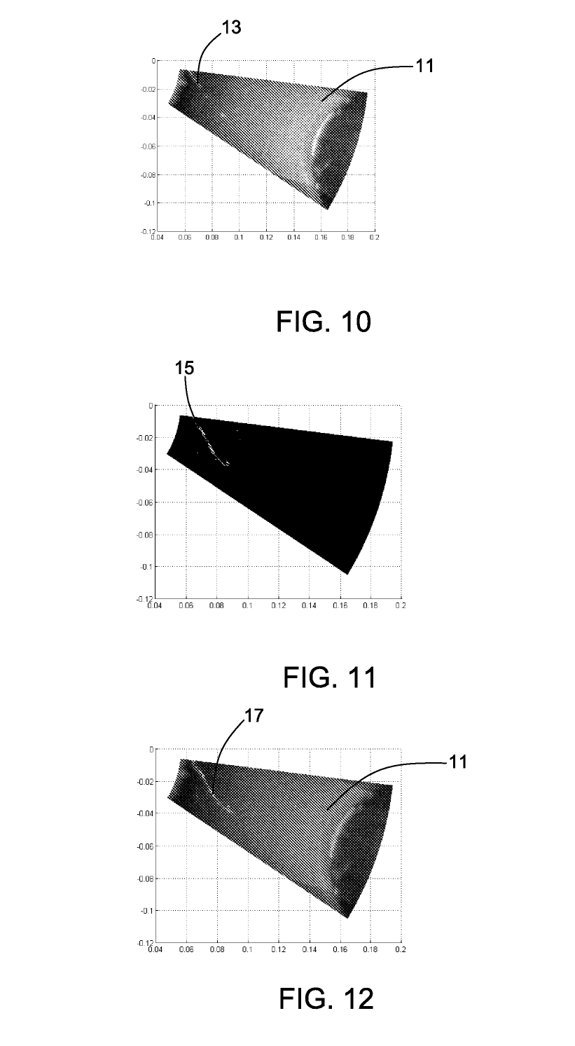

FIG. 10 illustrates a non-steered image in accordance with an embodiment. The non-steered image is obtained by processing a non-steered frame which may be the non-steered frame 401 of FIGS. 2 to 8. In the non-steered image, the structures 11 in the body are imaged clearly while the needle 13 in the body is not imaged clearly. For illustration purpose, the non-steered image is displayed as a visible image. In some embodiments, the not-steered image is not displayed, and may be stored in a memory.

FIG. 11 illustrates a needle-enhancement image in accordance with an embodiment. The needle-enhancement image is obtained by processing a steered frame which may be one of the steered frames 403, 410, 416, 420, 426, 436, 440, 446 in FIGS. 2 to 8. In the needle-enhancement image, only the needle 15 is imaged clearly and a background of the needle 15, in this embodiment, is black. For illustration purpose, the needle-enhancement image is displayed as a visible image. In some embodiments, the needle-enhancement image is not displayed, and may be stored in a memory. In certain embodiments, one or more needle-enhancement images are obtained using one or more steered frames.

FIG. 12 illustrates a compound image of the non-steered image of FIG. 10 and the needle-enhancement image of FIG. 11. The compound image is obtained by spatially compounding the non-steered image of FIG. 10 and the needle-enhancement image of FIG. 11. In the compound image, the needle 17 is obtained by compounding the needle 13 in FIG. 10 and the needle 15 in FIG. 11, and the structures 11 in FIG. 12 are the structures 11 in FIG. 11. The needle is enhanced and the structures 11 in the body are clear. The compound image is displayed by the display device 106 of FIG. 1.

While embodiments of the invention have been described herein, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

Furthermore, the skilled artisan will recognize the interchangeability of various features from different embodiments. The various features described, as well as other known equivalents for each feature, can be mixed and matched by one of ordinary skill in this art to construct additional systems and techniques in accordance with principles of this disclosure.

What is claimed is:

1. An apparatus, comprising:
a convex probe comprising a plurality of transducer elements configured to transmit a plurality of sets of ultrasound signals, each of the plurality of sets of ultrasound signals comprising a non-steered frame and at least one steered frame angled toward the non-steered frame;
a receiver coupled to the convex probe and configured to receive a non-steered echo frame for the non-steered fame and at least a steered echo frame for the at least one steered frame; and
an image generating device coupled to the receiver and configured to generate a non-steered image using the non-steered echo frame and at least one needle-enhancement image using the at least one steered echo frame, and further configured to combine the non-steered image and the at least one needle-enhancement image into a compound image.

2. The apparatus of claim 1, wherein the at least one steered frame comprises left steered beams steered leftward with respect to an axis of the convex probe and right steered beams steered rightward with respect to the axis of the convex probe.

3. The apparatus of claim 1, wherein the at least one steered frame comprises vertical steered beams parallel with an axis of the convex probe.

4. The apparatus of claim 1, wherein the at least one steered frame comprises steered beams steered in parallel.

5. The apparatus of claim 1, wherein the at least one steered frame comprises steered beams steered at different angles with respect to an axis of the convex probe.

6. The apparatus of claim 1, wherein the number of steered beams of the at least one steered frame is less than the number of non-steered beams of the non-steered frame.

7. The apparatus of claim 1, wherein the at least one steered frame comprises a left steered frame steered leftward with respect to an axis of the convex probe and a right steered frame steered rightward with respect to the axis of the convex probe.

8. The apparatus of claim 1, wherein the non-steered frame comprises non-steered beams transmitted at different angles with respect to an axis of the convex probe.

9. The apparatus of claim 1, wherein the image generating device comprises a steered image processor configured to generate at least one steered image using the at least one steered echo frame and a needle detection processor coupled to the steered image processor and configured to filter the at least one steered image to remove a non-needle image from the at least one steered image.

10. An apparatus, comprising:
a convex probe comprising a plurality of transducer elements configured to transmit a plurality of sets of ultrasound signals, each of the plurality of sets of ultrasound signals comprising a non-steered frame and at least one steered frame angled to the non-steered frame; and
a processing unit coupled to the convex probe and configured to:
receive ultrasound echo signals for each of the plurality of sets of ultrasound signals,
generate a non-steered image and at least one needle-enhancement image using the ultrasound echo signals, and
combine the non-steered image and the at least one needle-enhancement image into a compound image.

11. The apparatus of claim 10, wherein the at least one steered frame comprises left steered beams steered leftward with respect to an axis of the convex probe and right steered beams steered rightward with respect to the axis of the convex probe.

12. The apparatus of claim 10, wherein the at least one steered frame comprises vertical steered beams parallel with an axis of the convex probe.

13. The apparatus of claim 10, wherein the at least one steered frame comprises steered beams steered in parallel.

14. The apparatus of claim 10, wherein the at least one steered frame comprises steered beams steered at different angles with respect to an axis of the convex probe.

15. The apparatus of claim 10, wherein the number of steered beams of the at least a steered frame is less than the number of non-steered beams of the non-steered frame.

16. A method, comprising:
transmitting a plurality of sets of ultrasound signals from a plurality of transducer elements of a convex probe, each of the plurality of sets of ultrasound signals comprising a non-steered frame and at least one steered frame angled to the non-steered frame;
receiving a non-steered echo frame for the non-steered fame and at least one steered echo frame for the at least one steered frame;
generating a non-steered image using the non-steered echo frame and at least one needle-enhancement image using the at least one steered echo frame; and
combining the non-steered image and the at least one needle-enhancement image into a compound image.

17. The method of claim 16, wherein the at least one steered frame comprises left steered beams steered leftward with respect to an axis of the convex probe and right steered beams steered rightward with respect to the axis of the convex probe.

18. The method of claim 16, wherein the at least one steered frame comprises vertical steered beams parallel with an axis of the convex probe.

19. The method of claim 16, wherein the number of steered beams of the at least a steered frame is less than the number of non-steered beams of the non-steered frame.

20. The method of claim 16, wherein generating the at least one needle-enhancement image comprises generating at least one steered image using the at least one steered echo frame and selecting the at least one needle-enhancement image from the at least one steered image.
